(19) United States
(12) Patent Application Publication
(10) Pub. No.: US 2022/0298541 A1
(43) Pub. Date: Sep. 22, 2022

(54) ASSAY FOR DETERMINING TARGET ENGAGEMENT IN REAL-TIME IN LIVING SYSTEMS

(71) Applicant: Indian Institute of Science Education and Research, Pashan (IN)

(72) Inventors: Britto S. SANDANARAJ, Pashan (IN); Punita BATHLA, Pashan (IN); Abhijit DE, Mumbai (IN); Aaiyas MUJAWAR, Mumbai (IN)

(73) Assignee: Indian Institute of Science Education and Research, Pashan (IN)

(21) Appl. No.: 17/698,397

(22) Filed: Mar. 18, 2022

(30) Foreign Application Priority Data
Mar. 19, 2021 (IN) 202121011849

(51) Int. Cl. C12Q 1/66; G01N 33/58; G01N 33/50

(52) U.S. Cl. CPC C12Q 1/66; G01N 33/582; G01N 33/502; C07K 2319/60; G01N 2333/43595

(57) ABSTRACT

The present disclosure relates to an assay for determining drug-target engagement in real-time. Specifically, the present disclosure provides a method for determining drug candidate-target interaction in real-time in living systems using Activity-based Reporter Gene Technology-Bioluminescence Resonance Energy Transfer (AbRGT-BRET) based assay. The said assay is useful for High Throughput Screening of compounds to a specific target.

### RELATED APPLICATIONS

This application claims priority under 35 U.S.C. § 119(a) to Indian Patent Application No. 202121011849, filed Mar. 19, 2021, which application is incorporated herein by reference.

### FIELD OF THE INVENTION

The present disclosure pertains to a method for determining target engagement in living systems in real-time to facilitate drug discovery. In particular, the present disclosure provides a method for determining drug candidate-target interaction in real-time in living systems using Activity-based Reporter Gene Technology-Bioluminescence Resonance Energy Transfer (AbRGT-BRET) based assay.

### BACKGROUND OF THE INVENTION

The background description includes information that may be useful in understanding the present invention. It is not an admission that any of the information provided herein is prior art or relevant to the present invention, or that any publication specifically or implicitly referenced is prior art.

Drug-target interaction determination plays an important role in both drug discovery and development efforts. Development of robust assay platforms for studying drug-target interaction would not only facilitate the discovery of novel first-in-class medicines but also greatly reduce the cost associated with developing a drug from early preclinical studies to clinics (Hay M, Thomas D W, Craighead J L, Economides C, Rosenthal J. 2014. Clinical development success rates for investigational drugs. Nat. Biotechnol. 32:40-51; Smietana K, Siatkowski M, Moller M. 2016. Trends in clinical success rates. Nat. Rev. Drug Discov. 15:379-80). Many technologies have been developed over the last three decades. Most of the early technologies relied on studying drug-target interaction using recombinant proteins in vitro conditions and rank ordering of the compounds based on IC50 values. However, these assays failed during cellular phenotypic screening thus producing confounding results (Schwaid A G, Cornella-Taracido I. 2018. Causes and significance of increased compound potency in cellular or physiological contexts. J. Med. Chem. 61:1767-73; Renaud J P, Chung C W, Danielson U H, Egner U, Hennig M, et al. 2016. Biophysics in drug discovery:impact, challenges and opportunities. Nat. Rev. Drug Discov. 15:679-98).

Another method of the use of cell lysates instead of recombinant proteins for target validation studies had received considerable interest. In comparison to in vitro biochemical studies, cell lysate experiments are considered to be better suited for target validation because they partially mimic the physiological state of a target. However, in most of the experiments this method has failed to capture intended drug-target interactions. One of the major limitations of this approach is the dissolution of cell membranes and other organelles during lysate preparation and hence the target environment is far from the realistic situation (5. Bantscheff M, Eberhard D, Abraham Y, Bastuck S, Boesche M, et al. 2007. Quantitative chemical proteomics reveals mechanisms of action of clinical ABL kinase inhibitors. Nat. Biotechnol. 25:1035-44; Bantscheff M, Hopf C, Savitski M M, Dittmann A, Grandi P, et al. 2011. Chemoproteomics profiling of HDAC inhibitors reveals selective targeting of HDAC complexes. Nat. Biotechnol. 29:255-65; Klaeger S, Heinzlmeir S, Wilhelm M, Polzer H, Vick B, et al. 2017. The target landscape of clinical kinase drugs. Science 358:eaan4368; 13. Davis M I, Hunt J P, Herrgard S, Ciceri P, Wodicka LM, et al. 2011. Comprehensive analysis of kinase inhibitor selectivity. Nat. Biotechnol. 29:1046-51). Therefore, developing technologies to study drug-target interaction in live intact cells has gained enormous interest in recent years (15.Zhao Q, Ouyang X, Wan X, Gajiwala K S, Kath J C, et al. 2017. Broad-spectrum kinase profiling in live cells with lysine-targeted sulfonyl fluoride probes. J. Am. Chem. Soc. 139:680-85). Although the success rate with these technologies is slightly better compared to technologies based on cell-lysate experiments, the success rate has not been very satisfactory in number of true hits, hence requiring alternate approaches to improve the success rate.

Some of the recent technologies that are usually used in drug discovery efforts include Activity-based protein profiling (ABPP), and fluorescence-based assays. The merits of these technologies have been evaluated based on a list of parameters that includes physiological relevance of a target, through-put efficiency, target modification, quantification, real-time kinetics, detection requirement, selectivity level, cell-type bias, target class bias, and spatio-temporal analysis. However, each one of the methods has been found to have limitations in respect of one or more of the such parameters, in particular for ability to determine the drug-target interaction in real-time in living systems

Thus, the development of accurate methods for quantifying drug-target interaction in live cells is still far from true physiological conditions and therefore there is a need for the development of chemical biology technologies to determine drug-target interaction in cellular and animal models can be crucial in this field. Few techniques which are available are mostly carried out in ex vivo conditions in which target tissue is excised from the sacrificed animal and subjected to down-stream processing includes tissue homogenization and cell lysis followed by mass spec detection (Simon G M, Niphakis M J, Cravatt B F, 2013. Determining target engagement in living systems. Nat. Chem. Biol. 9:200-5; Adibekian, A. et al. J. Am. Chem. Soc. 134, 10345-10348 (2012). However, this technique does not offer a way to determine the drug-target interaction in the living systems in real-time. This is a one of the serious limitations considering the spatio-temporal change of drug-target interaction during the drug treatment. Besides, this method may not work for targets which are expressed in very low concentration. Low-throughput and highly labour-intensiveness are some of the other drawbacks of this method. Although, there are few imaging methods available to study drug-target interaction in live animals, most of them are based on indirect read out (i.e. quantifying biomarkers) and they require very sophisticated infra-structure and trained personnel to carry out those studies (Krishna, R., Herman, G. & Wagner, J. A. AAPS J. 10, 401-409 (2008); Wong, D. F., Tauscher, J. & Grunder, G. Neuropsychopharmacology 34, 187-203 (2009). Matthews, P. M., Rabiner, E. A., Passchier, J. & Gunn, R. N. Br. J. Clin. Pharmacol. 73, 175-186 (2012). De, A., Ray, P., Loening, A. M. & Gambhir, S. S. BRET3: a red-shifted bioluminescence resonance energy transfer (BRET)-based integrated platform for imaging protein-protein interactions from single live cells and living animals. The FASEB Journal 23, 2702-2709 (2009)).

Thus, the development of accurate methods for quantifying drug candidate-target engagement in living systems is still far from true physiological conditions. Therefore, in view of one or more shortcomings and disadvantages like through-put efficiency, target modification, quantification, real-time kinetics of the technologies in the existing art in the field of drug screening, there remains an unmet need for the development of chemical biology technology and means for determining drug candidate-target interaction in living systems in real-time and can be applicable for low expressing targets.

### OBJECTS OF THE INVENTION

An object of the present invention is to provide a method for determining drug-target engagement to facilitate drug discovery.

An object of the present invention is to provide a method for determining drug candidate-target interaction in real-time in living systems.

An object of the present invention is to provide a method, and system for detection of intracellular interactions between a drug candidate and a known cellular target.

An object of the present invention is to provide a method for determining drug candidate-target interaction in real-time in living systems using Activity-based Reporter Gene Technology - Bioluminescence Resonance Energy Transfer (AbRGT-BRET) based assay.

An object of the present invention is to provide a method for detecting and analysing the interaction of drug candidate and target in living systems using AbRGT-BRET based assay system comprising an enzyme of interest (EoI) tethered to a bioluminescent donor moiety, an activity-based fluorescent probe (ABFP) with appropriate warhead and fluorophore.

Yet another object of the present invention is to provide a recombinant plasmid vector encoding for the luciferase enzyme tagged to the enzyme of interest (EoI) for use in AbRGT-BRET based assay, wherein the plasmid expresses a fusion protein of EoI and luciferase enzyme.

Yet another object of the present invention is to provide a method for expressing the recombinant plasmid vector encoding the EoI tagged luciferase enzyme in a suitable cell line by transiently transfecting the suitable cells with said plasmid for use in AbRGT-BRET based assay.

Yet another object of the present invention is to provide a cell line stably transfected with plasmid vector encoding the EoI tagged luciferase enzyme for use in AbRGT-BRET based assay.

Yet another object of the present invention is to provide a kit for conducting AbRGT-BRET based assay.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

The present invention provides a method, and system for detection of intracellular interactions between a drug candidate and a known or unknown cellular target in living systems in real-time.

In an aspect, the present disclosure relates to a method for determining drug-target engagement to facilitate drug discovery.

In an aspect, the present disclosure relates to a method for determining drug candidate-target interaction in living systems.

In an aspect, the present disclosure relates to a method for determining drug candidate-target interaction in real-time in living systems.

In an aspect, the present disclosure relates to a method, and system for detection of intracellular interactions between a drug candidate and a known cellular target.

In an aspect, the present disclosure relates to a method for determining drug candidate-target interaction in real-time in living systems using Activity-based Reporter Gene Technology-Bioluminescence Resonance Energy Transfer (AbRGT-BRET) based assay.

In an aspect, the present disclosure relates to a method for to detect drug candidate-target interaction real-time in living systems using activity-based reporter gene technology (AbRGT) in combination with bioluminescence resonance energy transfer (BRET).

In an aspect, the present disclosure relates to a method for detecting and analysing the interaction of drug candidate and target in living systems using AbRGT-BRET based assay system comprising an enzyme of interest (EoI) tethered to a bioluminescent donor moiety, an activity-based fluorescent probe (ABFP) with appropriate warhead and fluorophore.

In another aspect, the present disclosure relates to an ABFP comprising;
(a) a fluorophore selected from the group comprising fluoromethyl ketone (FMK), (acyloxy) methyl ketone (AOMK), E-64, and Q-VD-OPh;
(b) a linker sequence selected from the group comprising VAD, DEVD, VD, and D; and
(c) a fluorescent acceptor moiety having absorption spectrum in the region ranging from 550-650 nm.

In another aspect, the present disclosure relates to a recombinant enzyme comprising a protease polypeptide chain linked to a bioluminescent donor moiety; wherein the enzyme construct is subjected to stimuli in a host cell followed by addition of a fluorescent moiety having acceptor activity, wherein the donor and acceptor moieties exhibit bioluminescence resonance energy transfer (BRET).

In another aspect, the present disclosure relates to a nucleotide construct comprising sequences encoding regulatory elements operably linked to a nucleotide sequence encoding the recombinant enzyme linked to a bioluminescent donor moiety for use in AbRGT-BRET based assay. Further, the nucleotide construct is inserted in a plasmid vector for expression in a host cell.

In another aspect, the present disclosure relates to a nucleotide construct comprising sequences encoding regulatory elements operably linked to a nucleotide sequence encoding the recombinant protease linked to a bioluminescent donor moiety for use in AbRGT-BRET based assay. Further, the nucleotide construct is inserted in a plasmid vector for expression in a host cell.

In another aspect, the present disclosure relates to a method for determining drug candidate-target interaction in real-time in living systems using AbRGT-BRET based assay comprises the steps of:
(a) providing a recombinant plasmid vector encoding for a luciferase enzyme tagged to an enzyme-of-interest (EoI);
(b) expressing the recombinant plasmid vector encoding the EoI tagged to luciferase in a stably transfected cells;
(c) subjecting the stably transfected cells comprising the recombinant plasmid vector encoding the EoI tagged luciferase to a stimulus for activation of EoI tagged to luciferase, wherein the stimulus is a drug candidate;
(d) incubating stably transfected cells expressing the active EoI tagged luciferase and other endogenous proteases with an activity-based fluorescent probe (ABFP), wherein the ABFP comprises an appropriate reactive group and a fluorophore;
(e) adding a luciferase substrate to cells resulting in generating instantaneous BRET (bioluminescence resonance energy transfer) pair between the labelled EoI and ABFP; and
(f) measuring the BRET read out thereby reporting the activity of the EoI.

In another aspect, the present disclosure relates to a method for determining drug candidate-target interaction in real-time in living systems using AbRGT-BRET based assay comprises the steps of:
(a) providing a recombinant plasmid vector encoding for a luciferase enzyme tagged to an protease-of-interest (PoI);
(b) expressing the recombinant plasmid vector encoding the PoI tagged to luciferase in a stably transfected cells;
(c) subjecting the stably transfected cells comprising the recombinant plasmid vector encoding the PoI tagged luciferase to a stimulus for activation of PoI tagged to luciferase, wherein the stimulus is a drug candidate;
(d) incubating stably transfected cells expressing the active PoI tagged luciferase and other endogenous proteases with an activity-based fluorescent probe (ABFP), wherein the ABFP comprises an appropriate reactive group and a fluorophore;
(e) adding a luciferase substrate to cells resulting in generating instantaneous BRET (bioluminescence resonance energy transfer) pair between the labelled PoI and ABFP; and
(f) measuring the BRET read out thereby reporting the activity of the PoI.

In yet another aspect, the present disclosure relates to a recombinant plasmid vector encoding for the luciferase enzyme tagged to the protease-of-interest (PoI) for use in AbRGT-BRET based assay, wherein the plasmid expresses a fusion protein of PoI and luciferase enzyme.

In yet another aspect, the present disclosure relates to a method for expressing the recombinant plasmid vector encoding the PoI tagged luciferase enzyme in a suitable cell line by transiently transfecting the suitable cells with said plasmid for use in AbRGT-BRET based assay.

In yet another aspect, the present disclosure relates to a cell line transiently transfected with recombinant plasmid vector encoding the PoI tagged luciferase enzyme for use in AbRGT-BRET based assay.

In yet another aspect, the present disclosure relates to a cell line stably transfected with recombinant plasmid vector encoding the PoI tagged luciferase enzyme for use in AbRGT-BRET based assay.

In yet another aspect, the present disclosure relates to a MCF-7 cell line stably transfected with recombinant plasmid vector encoding the PoI tagged luciferase enzyme for use in AbRGT-BRET based assay.

In yet another aspect, the present disclosure relates to a kit for conducting AbRGT-BRET based assay, wherein the kit comprises one or more of: a recombinant plasmid vector encoding for the luciferase enzyme tagged to the Enzyme-of-interest (EoI); a stimulus for activation of EoI tagged to luciferase; an activity-based fluorescent probe (ABFP) with appropriate reactive group and fluorophore; a luciferase substrate; and optionally other chemical biological reagents to aid in the assay and instructions to carry out the assay.

In yet another aspect, the present disclosure relates to a kit for conducting AbRGT-BRET based assay, wherein the kit comprises one or more of: a recombinant plasmid vector encoding for the luciferase enzyme tagged to the protease-of-interest (PoI); a stimulus for activation of PoI tagged to luciferase; an activity-based fluorescent probe (ABFP) comprising Rh-VAD-FMK probe; coelenterazine H; and optionally other chemical biological reagents to aid in the assay and instructions to carry out the assay.

Various objects, features, aspects and advantages of the inventive subject matter will become more apparent from the following detailed description of preferred embodiments.

### BRIEF DESCRIPTION OF DRAWINGS

Characteristics and advantages of the subject matter as disclosed in the present disclosure will become clearer from the detailed description of an embodiment thereof, with reference to the attached drawing, given purely by way of an example, in which:

FIG. 1 is a schematic representation of the Activity-based Reporter Gene Technology-Bioluminescence Resonance Energy Transfer (AbRGT-BRET) based assay.

FIG. 2 is a Schematic illustration of the spectral overlap between the normalized RLuc 8.6 emission spectra and the normalized excitation and emission spectra of the rhodamine dye.

FIG. 3 represents the image of Recombinant pCMV-GGS-RLuc 8.6-caspase-3 plasmid.

FIG. 4 is a bar graph representing the expression of recombinant pCMV-GGS-RLuc 8.6-caspase-3 (Casp-3 RLuc 8.6).

FIG. 5 is a bar graph representing the caspase-3 activation levels in Staurosporine (STS) treated MCF-7 cell line transfected with pCMV-caspase-3-GGSRLuc 8.6 plasmid.

FIG. 6 is a graph representing the effect of Z-VAD-FMK inhibitor on Caspase-3 activation.

FIG. 7 is a graph representing the BRET approach of AbRGT for screening inhibitors in a 96 well plate.

FIG. 8 is a bar graph representing the quantification of caspase-3 activation in Caspase-3 RLuc expressing stable MCF-7 clones.

FIG. 9 is a bar graph representing the levels of caspase activation by various concentrations of Staurosporine (STS) in Caspase-3 RLuc expressing stable MCF-7 clones.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments;

on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weight percentages, angles, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms are used herein. To the extent a term used in a claim is not defined below, it should be given the broadest definition persons in the pertinent art have given that term as reflected in printed publications and issued patents at the time of filing.

While a particular form of the invention has been illustrated and described, it will be apparent that various modifications can be made without departing from the spirit and scope of the invention.

The term “drug candidate” as used herein refers to a drug candidate to be studied or multiple drug candidates under development and to be screened. The “drug candidate” can be a small molecule. The “drug candidate” can be a biological molecule selected from but not limiting to a peptide, protein, antibodies, enzymes, blood component(s), nucleic acid-based molecules e.g., RNAi, and genes; cells and tissue for therapies or transplant; and others agents for eliciting desired response in the subject.

The present disclosure pertains to an assay for determining target engagement in real-time.

The present disclosure pertains to an assay for determining and quantifying drug candidate-target interaction in real-time in living systems.

In an embodiment, the present invention provides methods, and systems for detection of intracellular interactions between a drug candidate and a known cellular target.

In an embodiment, the present invention provides means and methods for determining and quantifying drug candidate-target interaction to facilitate drug discovery. In particular, provided herein are: an enzyme of interest (EoI) tethered to a bioluminescent donor moiety; an activity-based fluorescent probe (ABFP) with appropriate warhead and fluorophore; a substrate for bioluminescent donor moiety; and methods of detecting and analysing the interaction of drug candidate and target.

In an embodiment, the present invention provides means and methods for determining and quantifying drug candidate-target interaction to facilitate drug discovery. In particular, provided herein are: a protease of interest (PoI) tethered to luciferase; an activity-based fluorescent probe (ABFP) with appropriate warhead and fluorophore; a substrate for luciferase; and methods of detecting and analysing the interaction of drug candidate and target.

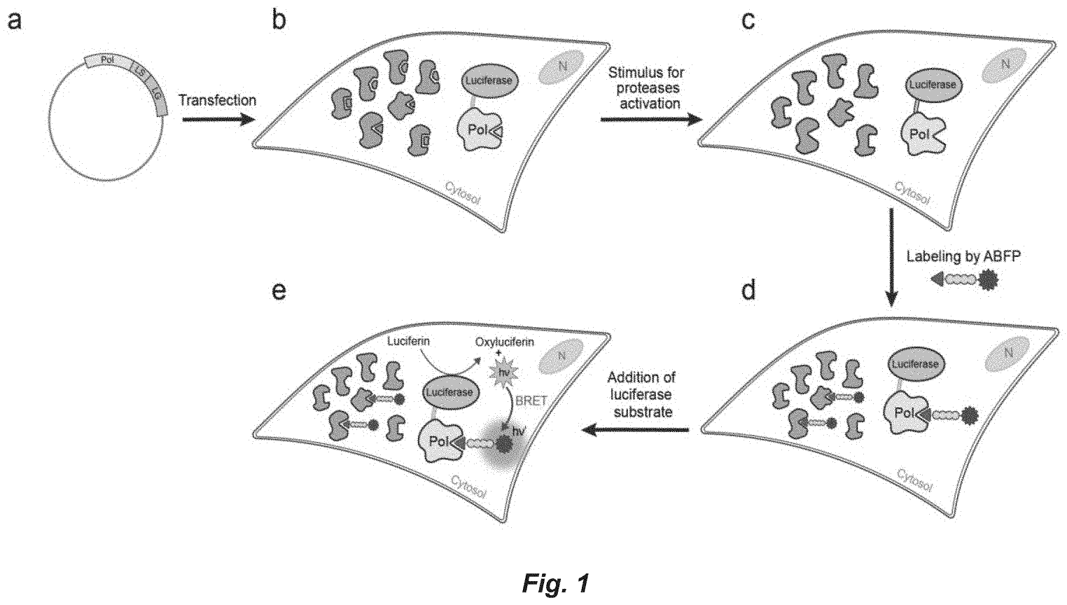

In an embodiment, the present disclosure provides an activity-based reporter gene technology (AbRGT), which detects the activity of an EoI (Enzyme of interest) in the present invention is schematically shown in FIG. 1.

In an embodiment, the present disclosure provides an activity-based reporter gene technology (AbRGT), which detects the activity of a PoI (Protease of interest) in the present invention is schematically shown in FIG. 1.

In an embodiment, the present disclosure provides a method for determining drug candidate-target interaction in real-time in living systems using an activity-based reporter gene technology (AbRGT) in combination with bioluminescence resonance energy transfer (BRET) system (hereafter referred to as AbRGT-BRET based assay).

In an embodiment of the present invention, the drug candidate is a potential therapeutic compound including but not limited to an agonist, an antagonist, naturally occurring molecule, synthetic molecule, pharmaceuticals, small molecule, macromolecule, nucleic acid, peptide, antibody, ligand, drug-like molecule, and the like.

In an embodiment of the present invention, the Bioluminescence Resonance Energy (BRET) system includes a BRET pair comprising a suitable donor and acceptor.

In certain embodiments of the present invention, the BRET system uses a fluorophore and bioluminescent donor moiety pair are selected that are sufficiently bright to allow detection of the transferred signal at a native abundance (or near native abundance) of the cellular target fused to the bioluminescent donor moiety.

In a preferred embodiment, the present invention provides a recombinant enzyme comprising a protease polypeptide chain linked to a bioluminescent donor moiety; wherein the enzyme construct is subjected to a stimuli in a host cell followed by the addition of a fluorescent moiety having acceptor activity, wherein the donor and acceptor moiety together form bioluminescence resonance energy transfer (BRET) pair.

In yet another preferred embodiment, the present invention provides a nucleotide construct comprising sequences encoding regulatory elements operably linked to a nucleotide sequence encoding the recombinant EoI linked to a bioluminescent donor moiety. Further, the nucleotide construct is inserted into a plasmid vector for expression in a host.

In yet another preferred embodiment, the present invention provides a nucleotide construct comprising sequences encoding regulatory elements operably linked to a nucleotide sequence encoding the recombinant PoI linked to luciferase. Further, the nucleotide construct is inserted into a plasmid vector for expression in a host.

In an embodiment of the present invention, the EoI or PoI and bioluminescent donor moiety are fused, tethered, or connected by expression as a fusion construct with or without peptide linker.

In an embodiment of the present invention, the EoI or PoI and bioluminescent donor moiety can be chemically linked, enzymatically linked, linked by a linker, and the like.

In an embodiment of the present invention, the living systems is a live cell, live plant or live animal.

In an embodiment of the present invention, the nucleotide construct of at least 70% sequence identity (e.g., 75% identity, 80% identity, 85% identity, 90% identity, 95% identity, 98% identity, 99% identity, or 100% identity) with the nucleotide construct of this invention.

In an embodiment of the present invention, the recombinant enzyme comprising a protease polypeptide chain linked to a bioluminescent donor moiety having a polypeptide of at least 70% sequence identity (e.g., 75% identity, 80% identity, 85% identity, 90% identity, 95% identity, 98% identity, 99% identity, or 100% identity) with the polypeptide of this invention.

In an embodiment, the present invention provides a transgene encoding a target protein, and a bioluminescent donor moiety, wherein the bioluminescent donor moiety comprises a Luciferase or variants available in prior-arts thereof.

In an embodiment of the present invention, the plasmid vectors, among others, includes: a polynucleotide sequence encoding a first target protein, and a bioluminescent donor moiety, wherein the bioluminescent donor moiety comprises a Luciferase or variants available in prior-arts thereof.

In an embodiment of the present invention, the luciferase is selected from Cypridina noctiluca-derived luciferase, Vargula hilgendorfii-derived luciferase, Oplophorus gracilirostris (Oplophoridae)-derived luciferase, Metridia longa (of Copepoda)-derived luciferase, Renilla-derived luciferase, fungal luciferase, firefly luciferase, click beetle luciferase, and the like. Most preferably Renilla-derived luciferase.

In an embodiment of the present invention, the substrate for luciferase include but not limited to coelenterazine, a coelenterazine derivative, a molecule substantially equivalent to coelenterazine, a structural or functional equivalent of coelenterazine, or a molecule functionally or structurally similar to coelenterazine. Most preferably coelenterazine h or v.

In an embodiment of the present invention, the substrate for luciferase include but not limited to luciferin, a luciferin derivative, a molecule substantially equivalent to luciferin, a structural or functional equivalent of luciferin, or a molecule functionally or structurally similar to luciferin.

In an embodiment of the present invention, the luciferase catalyses oxidation of luciferin, (substrate), with oxygen molecules and the energy that has been produced during the reaction causes the generation of an oxidation product (oxyluciferin) at an excited state, which on returning to ground state causes luminescence.

In an embodiment of the present invention, the bioluminescent donor moiety converts the coelenterazine, coelenterazine derivative, structural or functional equivalent of coelenterazine, or substantial equivalent to coelenterazine into coelenteramide, a coelenteramide derivative, a structural or functional equivalent of coelenteramide, or a substantial equivalent to coelenteramide and releases light energy as a by-product, thereby leading to the generation of photons, and said photons are transferred to the BRET acceptor moiety or activity-based fluorescent probe (ABFP), and causing measurable excitation of ABFP.

In an embodiment of the present invention, the bioluminescent donor moiety converts luciferin. luciferin derivative, structural or functional equivalent of luciferin, or substantial equivalent to luciferin into oxyluciferin, a oxyluciferin derivative, a structural or functional equivalent of oxyluciferin, or a substantial equivalent to oxyluciferin and releases light energy as a by-product, thereby leading to the generation of photons, and said photons are transferred to the BRET acceptor moiety or activity-based fluorescent probe (ABFP), and causing measurable excitation of ABFP.

In an embodiment of the present invention, the BRET acceptor moiety (also known as ABFP) depends on the warhead and fluorescent tag.

In an embodiment of the present invention, the ABFP used is such that it allows a significant spectral overlapping between own excitation and the emission by the luciferase enzyme.

In an embodiment of the present invention, the BRET donor moiety is a dye having an emission spectrum of at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm. Most preferably in the region ranging from 500 nm to about 600 nm.

In an embodiment of the present invention, the fluorescent tag used in ABFP is a dye having an absorption spectrum of at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm. Most preferably in the region ranging from 550 nm to about 650 nm.

In an embodiment of the present invention, the fluorescent tag used in ABFP is selected from but not limited to the dyes comprising an absorption range of 550-650 nm including but not limited to alexafluor dyes, cyanine dyes, rhodamine, and the like. Most preferably rhodamine.

In an embodiment, the present invention provides an ABFP comprising;
(a) a fluorophore selected from the group comprising fluoromethyl ketone (FMK), (acyloxy) methyl ketone (AOMK), E-64, and Q-VD-OPh;
(b) a linker sequence selected from the group comprising VAD, DEVD, VD, and D; and
(c) a fluorescent acceptor moiety selected from the group of dyes having absorption spectrum in the region ranging from 550-650 nm.

In an embodiment, the present invention provides an ABFP comprising;
(d) a fluorophore selected from the group comprising fluoromethyl ketone (FMK), (acyloxy) methyl ketone (AOMK), E-64, and Q-VD-OPh;
(e) a linker sequence selected from the group comprising VAD, DEVD, VD, and D; and
(f) rhodamine, a fluorescent acceptor moiety having absorption spectrum in the region ranging from 550-650 nm.

In an embodiment of the present invention, the BRET pair includes luciferase as the BRET donor and rhodamine as the BRET acceptor, wherein luciferase is Renilla luciferase RLuc 8.6, an enzyme of 36 kDa that requires coelenterazine h as a substrate and emits at 535 nm wavelength; and rhodamine dye is excited at a wavelength of 561 nm, and its emission occurs at 590 nm.

In an embodiment of the present invention, the EoI or PoI is selected from but not limited to the group of proteases selected from but not limited to serine proteases, cysteine proteases, threonine proteases, aspartic proteases, glutamic proteases, metalloproteases, asparagine peptide lyases, caspases, and the like. Preferably caspases selected from selected from but not limited to caspase-3, -7, -8, -9; cathepsins selected from but not limited to cathepsin-B; or metalloproteases selected from MMP-2, -9, 14 and the like.

In an embodiment of the present invention, the stimulus for activation of the EoI or PoI tagged to luciferase is a potential a drug candidate under study including but not limited to an agonist, an antagonist, naturally occurring molecule, synthetic molecule, pharmaceuticals, small molecule, macromolecule, nucleic acid, peptide, antibody, ligand, drug-like molecule, and the like.

In an embodiment of the present invention, the promoter used in the plasmid may be constitutive or inducible. Constitutive promoters include, but are not limited to, immediate early cytomegalovirus (CMV) promoter, herpes simplex virus 1 (HSV1) immediate early promoter, SV40 promoter, lysozyme promoter, early and late CMV promoters, early and late HSV promoters, hormonal inducible promoter, beta-actin promoter, tubulin promoter, Rous-Sarcoma virus (RSV) promoter, and heat-shock protein (HSP) promoter. Inducible promoters include tissue-specific promoters, developmentally-regulated promoters and chemically inducible promoters. Examples of tissue-specific promoters include the glucose-6-phosphatase (G6P) promoter, vitellogenin promoter, ovalbumin promoter, ovomucoid promoter, conalbumin promoter, ovotransferrin promoter, prolactin promoter, kidney uromodulin promoter, and placental lactogen promoter. Examples of developmentally-regulated promoters include the homeobox promoters and several hormone induced promoters. Examples of chemically-inducible promoters include reproductive hormone induced promoters and antibiotic-inducible promoters such as the tetracycline-inducible promoter and the zinc-inducible metallothionine promoter. Such constructs can readily be employed to evaluate a library of promoter variants. Most preferably CMV promoter.

In an embodiment, the present disclosure provides an Activity-based Reporter Gene Technology-Bioluminescence Resonance Energy Transfer (AbRGT-BRET) based assay to determine drug candidate-target interaction in cellular and/or animal models.

In an embodiment, the present disclosure provides a method for determining drug candidate-target interaction in real-time in living systems using AbRGT-BRET based assay comprises the steps of
(a) providing a recombinant plasmid vector encoding for a luciferase enzyme tagged to an enzyme-of-interest (EoI);
(b) expressing the recombinant plasmid vector encoding the EoI tagged to luciferase in a stably transfected cells;
(c) subjecting the stably transfected cells comprising the recombinant plasmid vector encoding the EoI tagged luciferase to a stimulus for activation of EoI tagged to luciferase, wherein the stimulus is a drug candidate;
(d) incubating stably transfected cells expressing the active EoI tagged luciferase and other endogenous proteases with an activity-based fluorescent probe (ABFP), wherein the ABFP comprises an appropriate reactive group and a fluorophore;
(e) adding a luciferase substrate to cells resulting in generating instantaneous BRET (bioluminescence resonance energy transfer) pair between the labelled EoI and ABFP; and
(f) measuring the BRET read out thereby reporting the activity of the EoI.

In a preferred embodiment, the present disclosure provides a method for determining drug candidate-target interaction in real-time in living systems using AbRGT-BRET based assay comprises the steps of:
(a) providing a recombinant plasmid vector encoding for a luciferase enzyme tagged to an protease-of-interest (PoI);
(b) expressing the recombinant plasmid vector encoding the PoI tagged to luciferase in a stably transfected cells;
(c) subjecting the stably transfected cells comprising the recombinant plasmid vector encoding the PoI tagged luciferase to a stimulus for activation of PoI tagged to luciferase, wherein the stimulus is a drug candidate;
(d) incubating stably transfected cells expressing the active PoI tagged luciferase and other endogenous proteases with an activity-based fluorescent probe (ABFP), wherein the ABFP comprises an appropriate reactive group and a fluorophore;
(e) adding a luciferase substrate to cells resulting in generating instantaneous BRET (bioluminescence resonance energy transfer) pair between the labelled PoI and ABFP; and
(f) measuring the BRET read out thereby reporting the activity of the PoI.

In an embodiment of the present invention, the plasmid DNA include an Escherichia coli-derived plasmid (e.g., pBR, pUC or pBluescript), a Bacillus subtilis-derived plasmid (e.g., pUB or pTP), and a yeast-derived plasmid (e.g., a YEp system or an YCp system).

In an embodiment of the present invention, the plasmid can be a phagemid including but not limited to kphage (e.g., Charon series, EMBL series, λgt series or λZAP). In further embodiments, animal viruses such as retroviruses and vaccinia viruses, and insect virus vectors such as baculoviruses, can also be used.

In an embodiment, the present invention provides a recombinant plasmid vector comprising a luciferase-tagged protease-of interest (PoI) by cloning a protease-of interest (PoI) into a plasmid vector encoding for luciferase gene.

In an embodiment, the present invention provides a recombinant plasmid vector encoding for the luciferase enzyme tagged to the protease-of-interest (PoI), wherein the plasmid expresses a fusion protein of PoI and luciferase enzyme.

In an embodiment, the present invention provides a method for expressing the recombinant plasmid vector encoding the PoI tagged luciferase enzyme in a suitable cell line by transiently transfecting the suitable cells with said plasmid.

In an embodiment of the present invention, the transfection is performed by a method selected from but not limited to electroporation; viral vector-based methods (e.g.: adenovirus/adenoassociated/Lentivirus); non-viral methods (e.g.: naked DNA, gene gun or liposome or polymer based delivery vehicles); and the like.

In an embodiment, the present invention provides a method for expressing the recombinant plasmid vector encoding the PoI tagged luciferase enzyme in a suitable cell line by stably transfecting the suitable cells with said plasmid.

In an embodiment of the present invention, the suitable cell line is prokaryotic cell line, eukaryotic cell line or mammalian cell line such as such as MCF-7, CHO, VERO, BHK, HeLa, COS, MDCK, HEK293, NIH-3T3, WI38 cells and the like. Most preferably MCF-7 cells.

In an embodiment, the present invention provides a suitable cell line stably expressing PoI tagged luciferase enzyme can be implanted in animal model (preferably but not limited to mice, rat, hamster, guinea pigs, and the like), followed by the introduction of and suitable inhibitor or activator of the said POI, wherein the suitable concentration of ABFP and substrate of luciferase can be injected into the animal model, followed by measuring the activity of POI as BRET ratio.

In an embodiment of the present invention, the Activity-based Reporter Gene Technology-Bioluminescence Resonance Energy Transfer (AbRGT-BRET) based assay includes mBRET value calculation of the treated sampled with the drug candidate under study and untreated samples.

In a preferred embodiment of the present invention, the present disclosure provides a method for calculating mBRET values of the treated sampled with the drug candidate under study and untreated samples comprise the equation:

$$A / D channel emission ratio = \frac{Acceptor channel emission}{Donor channel emission} \\ \\ \\ BRET = \frac{Acceptor channel emission ( D + A )}{Donor channel emission ( D + A )} - \frac{Acceptor channel emission ( D only )}{Donor channel emission ( D only )} \\ mBRET unit = Corrected BRET ratio * 1000$$

In an embodiment of the present invention, the increase in mBRET values in the treated samples is an indication of the occurrence of BRET. The mBRET value is an indirect readout of the activation of the PoI. For example, if the drug candidate of interest under study is an inhibitor, the mBRET values drops in the presence of the inhibitor in the sample.

In an embodiment, the present disclosure provides a kit for conducting AbRGT-BRET based assay, wherein the kit comprises one or more of: a recombinant plasmid vector encoding for the BRET donor moiety tagged to the Enzyme-of-interest (EoI); a stimulus for activation of EoI tagged to BRET donor moiety; an activity-based fluorescent probe (ABFP) with appropriate reactive group and fluorophore; a luciferase substrate; and optionally other chemical biological reagents to aid in the assay and instructions to carry out the assay.

In another embodiment, the present disclosure provides a kit for conducting AbRGT-BRET based assay, wherein the kit comprises one or more of: a recombinant plasmid vector encoding for the luciferase enzyme tagged to the Enzyme-of-interest (EoI); a stimulus for activation of EoI tagged to luciferase; an activity-based fluorescent probe (ABFP) with appropriate reactive group and fluorophore; a luciferase substrate; and optionally other chemical biological reagents to aid in the assay and instructions to carry out the assay.

In a preferred embodiment, the present disclosure provides a kit for conducting AbRGT-BRET based assay, wherein the kit comprises one or more of: a recombinant plasmid vector encoding for the luciferase enzyme tagged to the protease-of-interest (PoI); a stimulus for activation of PoI tagged to luciferase; an activity-based fluorescent probe (ABFP) comprising Rh-VAD-FMK probe; coelenterazine H; and optionally other chemical biological reagents to aid in the assay and instructions to carry out the assay.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example 1: Choice of the BRET Donor and Acceptor

To develop the BRET approach of AbRGT, RLuc 8.6, and rhodamine was chosen as a BRET donor and acceptor, respectively. RLuc 8.6 is a 36 kDa enzyme that uses coelenterazine h as a substrate and emits at 535 nm wavelength. The rhodamine dye is excited at a wavelength of 561 nm, and the emission occurs at 590 nm.

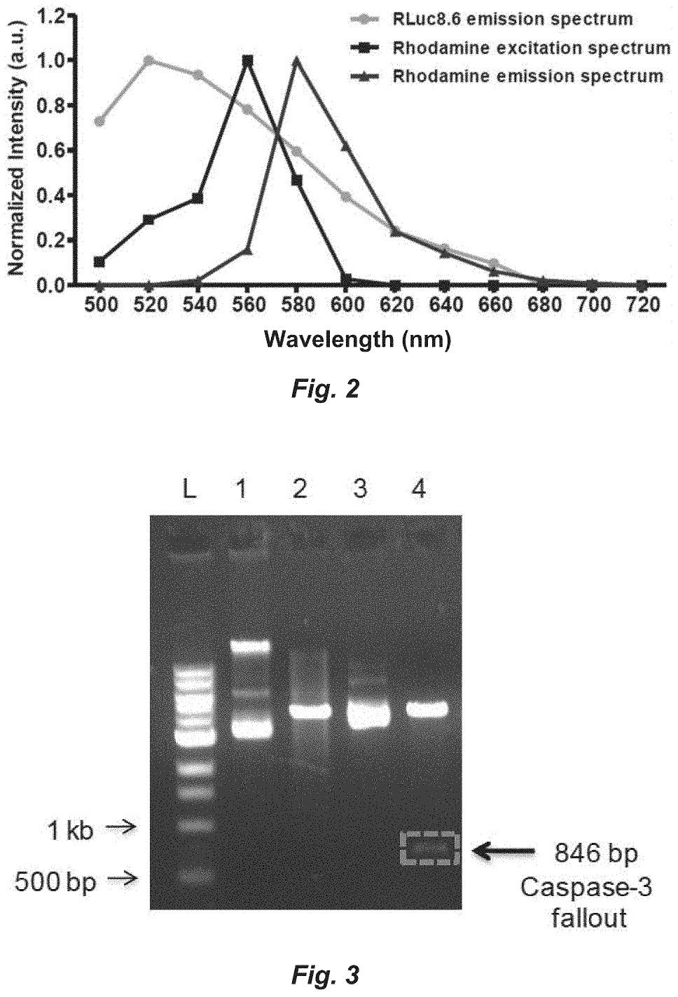

To determine the compatibility of RLuc 8.6 and rhodamine as a suitable BRET donor and acceptor, respectively, the spectral overlap between the two was analyzed. To obtain the RLuc 8.6 spectra, MCF-7 cells were transiently transfected with RLuc 8.6 plasmid. After 24 h of transfection, RLuc 8.6 substrate coelenterazine h was added to the cells. Immediately after substrate addition, the emission from RLuc is recorded from 500-700 nm. The emission maxima for the RLuc 8.6 was observed around 520-540 nm, consistent with the previous reports where the maxima are obtained at 535 nm. Afterwards, the excitation and emission spectra of rhodamine were acquired. To do that, we diluted the Rh-VAD-FMK probe in 1X PBS (Rh-VAD-FMK probe: 1X PBS, 1: 500 uL) and excited from 500-700 nm. The excitation maxima are observed at 561 nm, similar to the reported values. The values of RLuc 8.6 and rhodamine spectrum were normalized and merged to evaluate the spectral overlapping between the RLuc 8.6 emission and the rhodamine excitation. The significant spectral overlapping (540-600 nm) between the RLuc 8.6 and rhodamine was observed that establishes RLuc 8.6 and rhodamine as suitable BRET pair (FIG. 2)

### Example 2: Construction of Caspase-3 RLuc 8.6 Recombinant Vector and Cloning into pCMV-GGS-RLuc 8.6 Vectors

For caspase-3 RLuc 8.6 recombinant vector preparation, 846 bp of the human caspase-3 gene was PCR amplified from pCMV3-C-OFP spark plasmid using suitable primers. The amplified 846 bp caspase-3 gene was digested with Hind III and Bgl II restriction enzymes and cloned into pCMV-GGS-RLuc 8.6 vectors between the restriction sites Hind III and Bgl II using a PCR based method for restriction cloning. As can be seen from FIG. 3, Lane 1 represents undigested pCMV-GGS-RLuc 8.6 plasmid; Lane 2 represent pCMV-GGS-RLuc 8.6 plasmid digested with Hind III and Bgl II; Lane 3 is of undigested recombinant plasmid pCMV-GGS-RLuc 8.6-caspase-3; Lane 4 represents recombinant pCMV-GGS-RLuc 8.6-caspase-3 plasmid digested with Hind III and Bgl II showing 846 bp Caspase-3 fallout. (FIG. 3).

### Example 3: Expression of Caspase-3 RLuc 8.6 Vector in MCF-7 Cells

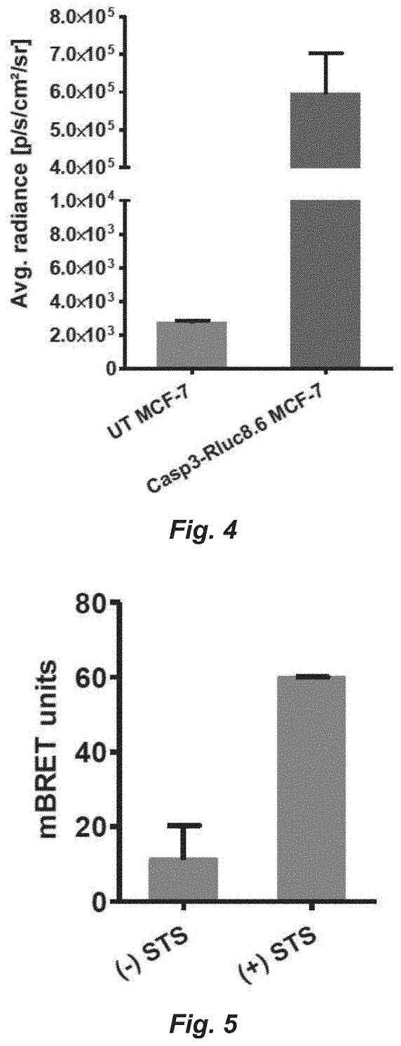

The expression of the recombinant caspase-3-GGS-RLuc 8.6 protein fusion was determined by transiently transfected MCF-7 cells with pCMV-caspase-3-GGSRLuc 8.6 plasmid. MCF-7 cells were seeded in a 35 mm dish and were transiently transfected with caspase-3 RLuc 8.6 recombinant plasmid at 70-80% cell confluency. 24 h post-transfection, the cells were trypsinized. The transfected cell were seeded in 96-black well plate (20000 cells/well) and 24h post-seeding to each well coelenterazine h was added and the untransfected (UT) MCF-7 cells added with coelenterazine h was used as negative control. Immediately after coelenterazine h addition, average radiance was recorded for both the untransfected and pCMV-caspase-3-GGSRLuc 8.6 transfected MCF-7 cells. The average radiance values for the caspase-3 RLuc transfected cells (5.9×10⁵ photons/sec/cm²/sr) is significantly higher as compared to the untransfected cells (2.6×10³ photons/sec/cm²/sr), confirming that the recombinant caspase-RLuc 8.6 plasmid is appropriately expressing in MCF-7 cells (FIG. 4).

### Example 4: Quantification of caspase-3 Activation using BRET-Approach of AbRGT

To determine the specific caspase-3 activation, pCMV-caspase-3-GGSRLuc 8.6 plasmid was overexpressed in the MCF-7 cell line since it is caspase-3 null. The cells were subjected to treatment with Staurosporine (STS) (1 μM) for 4 h for the induction of apoptosis. The STS triggers apoptosis via the intrinsic pathway and leads to the activation of caspases. Subsequently, cells were incubated with ABFP, Rh-VAD-FMK probe (BRET acceptor) for the labeling of PoI, and other proteases of the same family. The RLuc 8.6 substrate coelenterazine h was added to the cells, and the average radiance was recorded in the donor emission channel, RLuc 8.6 (520-540 nm), and the acceptor emission channel, rhodamine (580-600 nm) channel. The BRET measurements were done by calculating the mBRET values for the STS treated versus untreated samples using the following equation.⁷ Here, acceptor and donor is represented by (A) and (D), respectively.

$$A / D channel emission ratio = \frac{Acceptor channel emission}{Donor channel emission} \\ \\ \\ BRET = \frac{Acceptor channel emission ( D + A )}{Donor channel emission ( D + A )} - \frac{Acceptor channel emission ( D only )}{Donor channel emission ( D only )} \\ mBRET unit = Corrected BRET ratio * 1000$$

The calculated mBRET values for the STS treated sample is 60±0.4, which is significantly higher as compared to the untreated control, 11±9.2 (FIG. 4.5). This increase in mBRET values in the treated samples is an indication of the occurrence of BRET. The mBRET value is an indirect readout of the specific caspase-3 activation (FIG. 5).

### Example 5: Effect of Protease Inhibitor on STS Dependent Caspase Activation

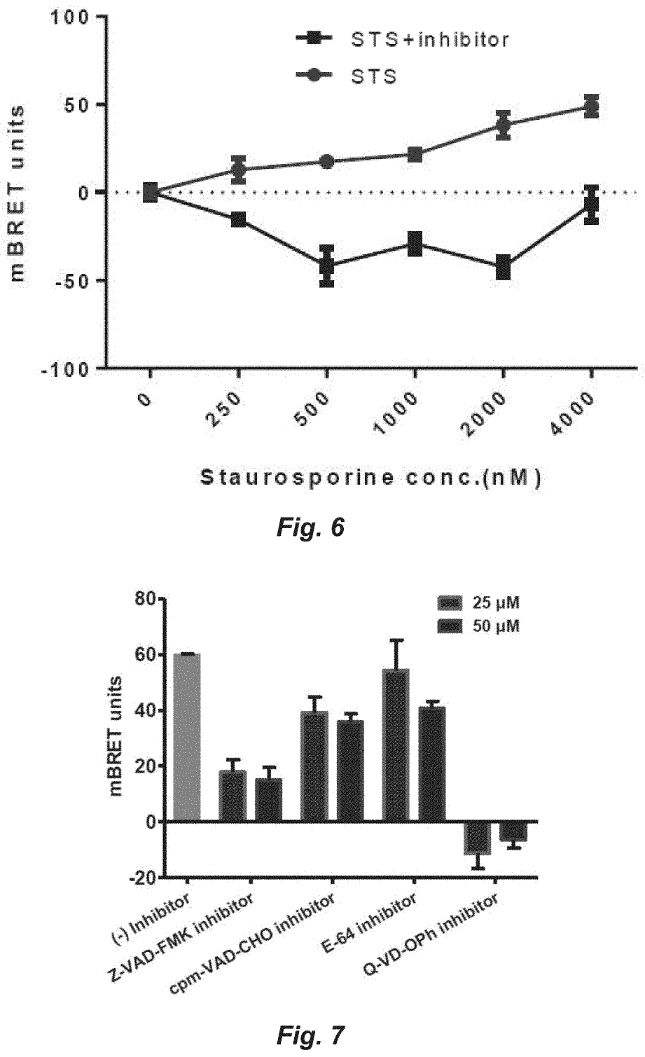

To check the effect of protease inhibitor Z-VAD-FMK on caspase activation, the pCMV-caspase-3-GGS-RLuc8.6 transiently transfected cells were treated with increasing concentration of STS(0-4 μM) followed by 1 h treatment of 50 μM of Z-VAD-FMK. The caspase3 activation was checked by BRET based AbRGT approach by using Rh-VAD-FMK probe. We found that the Z-VAD-FMK inhibits the caspase activation in presence of STS compared to only STS treated cells (FIG. 6). This data suggests that the BRET based approach can be applied to determine the activation as well as inhibition of caspase activity. Thus we can use the BRET-approach of AbRGT to screen caspase inhibitors and activators. (FIG. 6).

### Example 6: Screening Protease Inhibitors BRET-Approach of AbRGT

To show BRET-approach of AbRGT can be used for the high-throughput screening of inhibitors four different inhibitors; Z-VAD-FMK, C-inhibitor-II, E-64, and Q-VD-OPh were selected. Z-VAD-FMK, E-64, and Q-VD-OPh are irreversible inhibitors, while cpm-VAD-CHO is a reversible inhibitor. Z-VAD-FMK and Q-VD-OPh are widely known pan-caspase inhibitors that inhibit caspases with a different mechanism. E-64 is a cysteine peptidase inhibitor, while caspase-inhibitor II reversibly inhibits caspases.

For inhibitor screening in a high-throughput format, caspase-3 RLuc transfected MCF-7 cells in 96 well plates were treated with STS (1 μM) for 4 h. Subsequently, after STS treatment, cells were incubated with Z-VAD-FMK or E64 or Q-VD-OPh inhibitors at two different concentrations (25 or 50 μM) for 1 h. Cells were then labeled with the Rh-VAD-FMK probe for 1 h, followed by the addition of coelenterazine h. Immediately after coelenterazine h addition, average radiance was recorded in the donor emission and the acceptor emission channel. The BRET measurements were done by calculating the mBRET values of each sample. The calculated mBRET value of the sample, in the absence of inhibitor, is 60±0.4. The mBRET values dropped in the presence of the inhibitor in the sample. The mBRET values are higher for Q-VD-OPh inhibitor as compared to Z-VAD-FMK inhibitor (Q-VD-OPh>Z-VAD-FMK) (FIG. 7).

### Example 7: Quantification of Caspase-3 Activation in Caspase-3 RLuc Expressing Stable MCF-7 Clones

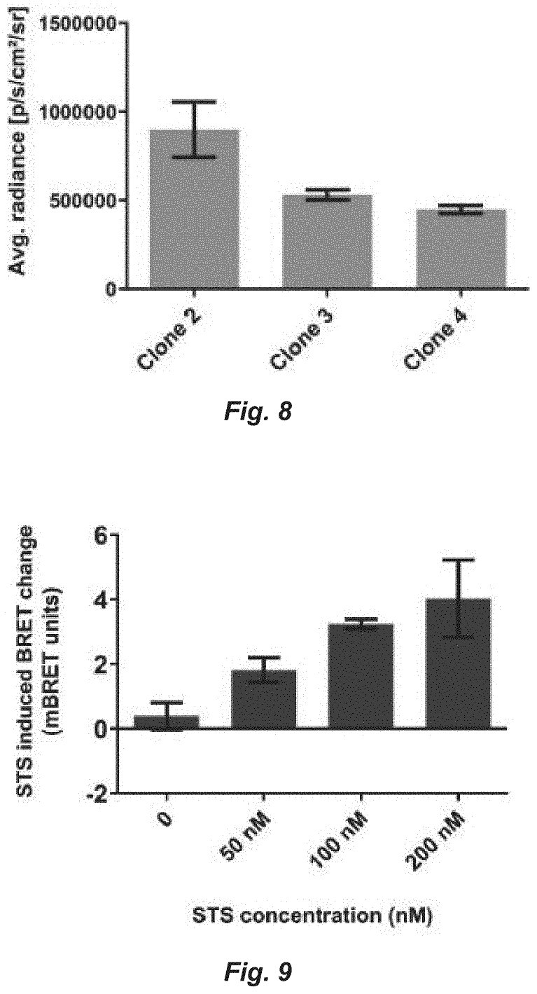

Luciferase activity in the MCF7 Caspase3-GGS-RLuc8.6 clone no. 2, 3, 4 was measured by the addition of luciferase substrate, coelentrazine. Immediately after substrate addition, average radiance value was recorded and plotted as a bar graph (FIG. 4.8). The MCF7 Caspase3-GGS-RLuc8.6 clone no. 2 cells shows the highest luciferase activity (˜1 ×10⁶ photons/sec/cm²/sr) and is selected for further experiments (FIG. 8).

The caspase-3 activity in the clone 2 cells further quantified by the BRET-approach of AbRGT. MCF-7 Caspase3-GGS-RLuc8.6 clone no. 2 cells were treated with increasing concentration (0, 50, 100, and 200 nM) of STS for 4 h. Subsequently, cells were incubated with Rh-VAD-FMK probe for 1 h followed by the addition of coelentrazine h. Immediately after substrate addition, average radiance was recorded at donor and acceptor filters and the mBRET values were calculated and plotted as a bar graph (FIG. 9).

An increase in mBRET values found with increasing STS concentration. A ˜4-fold increase in mBRET was found at 200 nM of STS concentration as compared to the untreated cells. This confirms the occurrence of BRET between RLuc 8.6 and Rhodamine of the Rh-VAD-FMK probe and activation of caspase-3 upon STS treatment. These results show the potential of the BRET-approach of AbRGT for the quantification of protease activation and for the high-throughput screening of protease inhibitors in cells.

Various modification and variation of the described assays, techniques and various means disclosed herein to implement the assays/methods in accordance with the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for evaluating drug candidate-target interaction in real-time by using activity-based reporter gene technology (AbRGT) comprising the steps of;
a. providing a recombinant plasmid vector encoding for a luciferase enzyme tagged to an enzyme-of-interest (EoI);
b. expressing the recombinant plasmid vector encoding the luciferase-tagged EoI in a stably transfected cells;
c. subjecting the stably transfected cells comprising the recombinant plasmid vector encoding the EoI tagged luciferase to a stimulus for activation of luciferase-tagged EoI, wherein the stimulus is a drug candidate;
d. incubating stably transfected cells expressing the active luciferase-tagged EoI and other endogenous proteases with an activity-based fluorescent probe (ABFP), wherein the ABFP comprises an appropriate reactive group and a fluorophore;
e. adding a luciferase substrate to cells resulting in generating instantaneous BRET (bioluminescence resonance energy transfer) pair between the labelled EoI and ABFP; and
f. measuring the BRET read out thereby reporting the activity of the EoI. characterized in that:
the assay evaluates drug candidate-target interaction in living systems; and
the AbRGT used in combination with BRET (AbRGT-BRET based assay) to detect drug-target interaction in real-time.

2. The method as claimed in claim 1, wherein the assay is used for drug-discovery.

3. The method as claimed in claim 1, wherein the recombinant plasmid vector comprises a nucleotide construct comprising sequences encoding regulatory elements operably linked to a nucleotide sequence encoding the recombinant EoI linked to luciferase.

4. The method as claimed in claim 1, wherein the drug candidate is a potential therapeutic compound comprising an agonist, an antagonist, naturally occurring molecule, synthetic molecule, pharmaceuticals, small molecule, macromolecule, nucleic acid, peptide, antibody, ligand, drug-like molecule, or combinations thereof.

5. The method as claimed in claim 1, wherein the EoI is a protease.

6. The method as claimed in claim 1, wherein the luciferase is Renilla Luciferase.

7. The method as claimed in claim 1, wherein the Renilla Luciferase is RLuc 8.6.

8. The method as claimed in claim 1, wherein the activity-based fluorescent probe moiety is a dye having an absorption spectrum in the region ranging from 550-650 nm.

9. The method as claimed in claim 1, wherein the ABFP comprises (a) a fluorophore selected from the group comprising of fluoromethyl ketone (FMK); (b) a linker sequence selected from the group comprising of VAD and DEVD; and (c) a fluorescent acceptor moiety which is a dye having an absorption spectrum in the region ranging from 550-650 nm.

10. The method as claimed in claim 7, wherein the ABFP comprises Z-VAD-FMK, C-inhibitor-II, E-64, or Q-VD-OPh

11. The method as claimed in claim 4, wherein the dye having an absorption spectrum in the region ranging from 550-650 nm is rhodamine dye.

12. The method as claimed in claim 1, wherein the substrate for the luciferase is luciferin, a luciferin derivative thereof, coelenterazine or a coelenterazine derivative thereof.

13. The method as claimed in claim 1, wherein upon activation of the Enzyme-of-Interest by the drug target, conversion of the luciferase substrate to a reaction product by the luciferase results in excitation of the ABFP by BRET and fluorescence emission from the ABFP.

14. A fusion protein comprising EoI linked to luciferase, wherein the fusion protein is expressed by the recombinant plasmid vector as claimed in claim 3.

15. An expression system comprising the recombinant plasmid vector as claimed in claim 3, wherein the expression system is a cell.

16. An expression system for performing AbRGT-BRET based assay as claimed in claim 1, wherein the expression system is a cell.

17. The cell as claimed in claim 14, wherein the cell is a transiently transfected cell or a stably transfected cell expressing the luciferase-tagged EoI.

18. The cell as claimed in claim 15, wherein the cell is a transiently transfected cell or a stably transfected cell expressing the luciferase-tagged EoI.

19. An animal model for performing AbRGT-BRET based assay as claimed in claim 1, wherein the animal model is implanted with the cell expressing the luciferase-tagged EoI as claimed in claim 16.

20. A kit for performing the AbRGT-BRET based assay as claimed in claim 1.

21. A kit for performing AbRGT-BRET based assay for identifying drug-target interactions, wherein the kit comprises
a. one or more of: a recombinant plasmid vector encoding for the luciferase enzyme tagged to the Enzyme-of-interest (EoI);
b. a recombinant plasmid vector encoding the luciferase-tagged EoI
c. a stimulus for activation of EoI tagged to luciferase, wherein the stimulus is a therapeutic agent;
d. a cell for expressing luciferase-tagged EoI;
e. an activity-based fluorescent probe (ABFP) with appropriate reactive group and fluorophore;
f. a luciferase substrate;
g. optionally any other chemical biological reagents to aid in the assay; and
h. instructions to carry out the assay.
